(19) United States
(12) Patent Application Publication
(10) Pub. No.: US 2026/0263157 A1
(43) Pub. Date: Sep. 10, 2026

(54) ARTIFICIAL INTELLIGENCE SENTINEL FOR SURGICAL PLANNING

(71) Applicant: Alcon Inc., Fribourg (CH)

(72) Inventors: Sinchan Bhattacharya, Denton, TX (US); Bryan Stanfill, Mansfield, TX (US); Shruti Siva Kumar, Foothill Ranch, CA (US); Praneeth Kurpad Narayanamurthy, Arlington, TX (US); Ramesh Sarangapani, Coppell, TX (US); Lu Yin, Keller, TX (US); Kevin Baker, Duluth, GA (US)

(21) Appl. No.: 19/555,472

(22) Filed: Mar. 3, 2026

Related U.S. Application Data
(60) Provisional application No. 63/766,789, filed on Mar. 4, 2025.

(51) Int. Cl. A61B 34/10; G16H 10/60; G16H 50/20

(52) U.S. Cl. CPC A61B 34/10; G16H 10/60; G16H 50/20

(57) ABSTRACT

A computer-implemented method of machine learning based surgical planning optimization. Embodiments include receiving, by an artificial intelligence agent, a request related to a surgical procedure that is to be performed on a patient. Embodiments include retrieving medical data that is related to the request from one or more source devices. Embodiments include generating, using a machine learning model, content related to the surgical procedure that is to be performed on the patient based on the request and the medical data, wherein the content comprises a set of relevant data points about the patient with respect to the surgical procedure or an indication of an issue related to a surgical plan. Embodiments include providing the content via an output device.

### CROSS-REFERENCE TO RELATED APPLICATIONS

This application claims the benefit of U.S. Provisional Application Serial No. 63/766,789 (filed on Mar. 4, 2025), the content of which is incorporated by reference herein in its entirety.

### INTRODUCTION

In a typical operating room or other medical treatment context, there is a vast amount of data being generated and utilized. This may include information from medical equipment, human communication, pre-operative data, human movement, and the like.

Leveraging such data in order to automatically generate useful recommendations or other content for use by medical professionals in connection with surgical planning can be challenging. For example, given the varying types, modalities, formats, and other attributes of such data, it is difficult to automatically determine which data is relevant to a particular context and/or to automatically analyze such data in a relational or holistic manner. Furthermore, given the large quantity of such data that may be available, it is technically difficult to process such data in a meaningful way without significant manual labor (e.g., to locate, curate, prepare, and/or otherwise analyze such data in a way that allows for meaningful analysis in connection with surgical planning). Given these technical challenges, automated recommendations or other content related to surgical planning that is generated based on such data using existing techniques may be inaccurate, may not be contextually informed, and/or otherwise may have limited utility.

Accordingly, there is a need for improved techniques for automated analysis and content generation based on disparate data sources related to surgical planning.

### SUMMARY

In certain embodiments, one general aspect includes a computer-implemented method for machine learning based surgical planning optimization. The computer-implemented method includes: receiving, by an artificial intelligence agent, a request related to a surgical procedure that is to be performed on a patient; retrieving medical data that is related to the request from one or more source devices; generating, using a machine learning model, content related to the surgical procedure that is to be performed on the patient based on the request and the medical data, wherein the content comprises a set of relevant data points about the patient with respect to the surgical procedure or an indication of an issue related to a surgical plan; and providing the content via an output device.

In certain embodiments, another general aspect includes a system. The system includes a memory having executable instructions and a processor in communication with the memory. The processor is configured to execute the instructions to perform the computer-implemented method for machine learning based medical treatment optimization described above.

In certain embodiments, another general aspect includes a computer-program product including a non-transitory computer-usable medium having computer-readable program code embodied therein. The computer-readable program code is adapted to be executed to implement the computer-implemented method for machine learning based medical treatment optimization described above.

### BRIEF DESCRIPTION OF THE DRAWINGS

FIG. 1 illustrates an example of computing components related to machine learning based medical treatment optimization, in accordance with certain embodiments of the present disclosure.

FIG. 2 illustrates an example process related to training a machine learning model for machine learning based surgical planning optimization, in accordance with certain embodiments of the present disclosure.

FIG. 3 illustrates an example of an artificial intelligence agent for machine learning based surgical planning optimization, in accordance with certain embodiments of the present disclosure.

FIG. 4 illustrates an example related to machine learning based surgical planning optimization, in accordance with certain embodiments of the present disclosure.

FIG. 5 illustrates another example related to machine learning based surgical planning optimization, in accordance with certain embodiments of the present disclosure.

FIG. 6 illustrates an examples of a process related to machine learning based surgical planning optimization, in accordance with certain embodiments of the present disclosure.

FIG. 7 illustrates an example of a computing device for machine learning based surgical planning optimization, in accordance with certain embodiments of the present disclosure.

### DETAILED DESCRIPTION

Large amounts of data are generated and stored in connection with treatment of patients in medical contexts, such as in connection with surgeries and other procedures. This data may be captured, generated, and/or stored by a variety of different devices, in different formats, for different purposes, and in different modalities such as text, images, video, audio, metadata, and the like.

Aspects of the present disclosure enable use of such various types of data for automated analysis and generation of accurate content such as relevant data and/or automatically detected issues related to surgical planning through the use of one or more particular artificial intelligence (AI) agents. According to certain aspects, an AI agent (which may be referred to as an AI “sentinel”) may utilize a machine learning model such as a large language model (LLM) to automatically generate outputs such as relevant data, automatically identified issues, or other types of content related to surgical planning to provide to medical professionals based on various types of input medical data (e.g., having different types and/or modalities).

For example, an AI agent may be used to screen pre-treatment measurements from diagnostics instruments, associated surgical planning documents, and metadata from electronic medical records (EMR) systems for patients scheduled for ophthalmological surgical procedures, including, but not limited to cataract surgery, a minimally invasive glaucoma surgery (MIGS), a vitreo-retinal surgery, or an intravitreal drug administration to identify potential documentation error, inconsistencies in data entry, potential error in intraocular lens (IOL) power calculations and/or toric power calculations, errors in intraoperative data, and/or other associated medical errors that can influence surgical outcomes.

An AI agent configured according to techniques described herein may retrieve data that is relevant to a particular request (e.g., for information or analysis related to surgical planning) from a variety of data sources. Such data may include, for instance, patient medical data, measurements from particular devices, information about a relevant surgical procedure, information about applicable devices and/or other items, information about operating rooms, inventory information, surgical plan documents, and/or the like. The AI agent may then provide such data to a machine learning model, such as along with a prompt instructing the machine learning model to output particular content (e.g., a listing of pertinent information, an indication of any issues related to a surgical plan, and/or the like) based on the data. The machine learning model may have been trained based on data about past surgical procedures, such as indicating surgical outcomes for patients having particular characteristics under particular contextual circumstances and/or when surgical plans represented by particular surgical plan documents were used. In some cases, multiple AI agents may utilize machine learning models of different types and/or trained using different techniques (e.g., supervised learning, unsupervised learning, or semi-supervised learning techniques) to generate content related to surgical planning based on relevant data.

Content generated by an AI agent may be provided via a user interface, such as in the form of text, image, video, and/or audio data that provides useful information to a medical professional with respect to surgical planning. For example, the content may include a listing of relevant information to use in preparing for a surgical procedure, such as the attributes of a patient and/or other contextual information that is particularly relevant to a given surgical procedure. In another example, the content may include an indication of one or more issues related to a surgical plan that were automatically identified by an AI agent, such as particular patient attributes or contextual conditions that may suggest an issue related to a surgical procedure (e.g., which may have been identified by a machine learning model as a result of being trained based on past associations between such patient attributes and/or contextual conditions and certain surgical outcomes). Such content may enable the development of an improved surgical plan that avoids issues and produces a better surgical outcome.

Embodiments of the present disclosure accomplish various technical improvements. For example, utilizing machine learning techniques described herein to automatically generate content related to surgical planning based on various types of data overcomes technical challenges associated with automated analysis of such data by enabling relationships among such data and relevant aspects of such data to be automatically identified and/or analyzed despite the varying modalities, formats, and types of such data and/or the quantity of such data. Deploying an AI agent described herein in a surgical setting enables live, interactive assistance to be automatically provided to medical professionals in connection with surgical planning based on information captured by various medical devices, sensors, and/or the like, such as allowing for automatically generating responses to natural language requests with a higher level of accuracy and utility than would be possible with existing techniques. In some cases, a user of systems implementing techniques described herein may be enabled to request automated assistance with surgical planning using intuitive natural language queries, and requested content (e.g., relevant patient information, results of an automated analysis of a surgical plan, and/or the like) may be automatically generated in an accurate manner based on a variety of different underlying data sources.

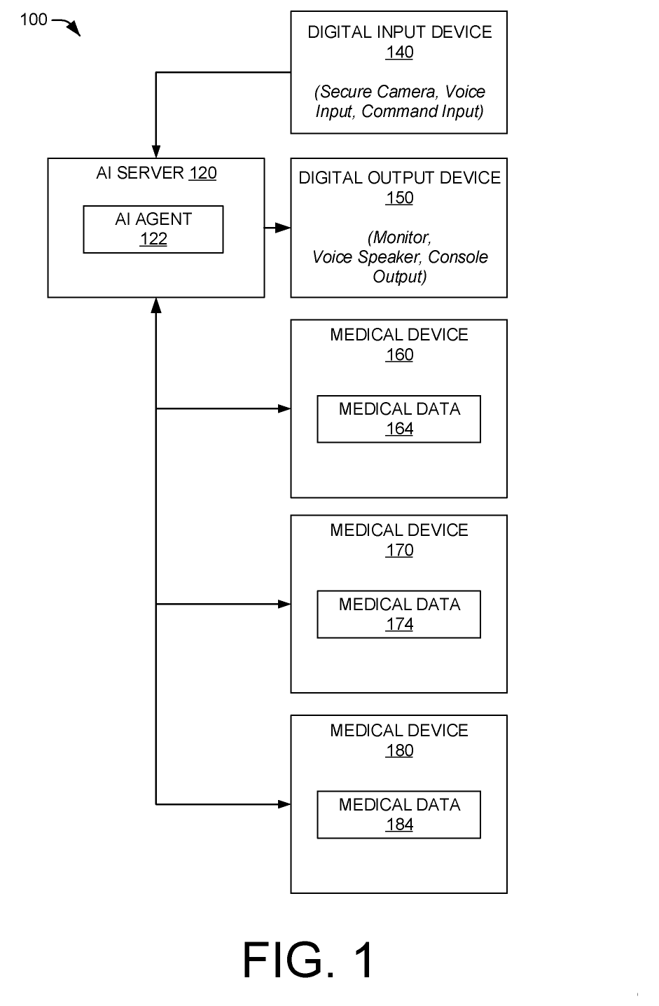

FIG. 1 illustrates an example computing environment 100 comprising computing components related to machine learning based surgical planning optimization, in accordance with certain embodiments of the present disclosure.

In computing environment 100, an artificial intelligence (AI) server 120 running an AI agent 122 is connected to a plurality of devices such as a digital input device 140, a digital output device 150, and medical devices 160, 170, and 180, such as via a network, which may be a wireless or wired connection (e.g., any type of connection over which data may be transmitted).

AI server 120 may be located in a clinical facility. In some aspects, AI server 120 is a physical or virtual computing device that runs AI agent 122 and utilizes digital input device 140 and digital output device 150 to receive requests from one or more users and provide requested content in response to such requests, such as based on using a machine learning model to automatically generate the requested content based on data from medical devices 160, 170, and/or 180. Digital input device 140 may include, for example, one or more devices such as a camera, microphone, mouse, keyboard, touch screen, and/or the like that enable receiving visual input (e.g., images and/or video), audio input, touch input, click input, text input, and/or the like. Digital output device 150 may include, for example, one or more devices such as a monitor or other screen, speaker, and/or the like for providing outputs in the form of text, images, video, sound, and/or the like.

Each of medical devices 160, 170, and 180 may be representative of a device capable of capturing, generating, and/or storing data related to clinical treatment of a patient. For example, medical devices 160, 170, and 180 may include one or more diagnostic instruments, ventilators, surgical instruments, health monitoring devices, activity monitoring devices, cameras, sensors, medical data storage components, electronic medical records (EMR) systems, and/or the like. Each of medical devices 160, 170, and 180 may comprise medical data 164, 174, or 184, which may include, for instance, data related to one or more patients, such as medical history data, test results, sensor data, treatment information, personal attributes, and/or the like. In some aspects, medical data 164, 174, or 184 may include device information such as state information, metadata, and/or the like. Medical data 164, 174, and 184 from medical devices 160, 170, and 180 may be provided to AI server 120 for automated analysis by AI agent 122. For example, medical data 164, 174, and 184 may be provided to AI server 120 at regular intervals, upon request from AI server 120, when one or more other conditions occur, and/or the like.

AI agent 122 may perform automated analysis of data (e.g., medical data 164, 174, and/or 184), such as based on one or more requests received via digital input device 140 and/or without such a request, in order to generate content related to surgical planning. In one example, a medical professional provides a request via digital input device 140 (e.g., via text, voice, video, and/or the like) for relevant patient information for a surgical procedure or for automated analysis of a surgical plan, and AI agent 122 retrieves data related to the request. For example, the data may include a subset of medical data 164, 174, and/or 184 that relates to a patient and/or procedure associated with the request. The data that is retrieved may include, for example, medical history data, measured health data, movement information, data about a procedure or medical condition, patient attributes, a surgical plan document, device state information, item inventory information, operating room availability information, and/or the like. Patient attributes may include information about a patient, such as personal characteristics (e.g., age, gender, and/or the like), medical history (e.g., known medical conditions, information about the extent of known medical conditions, procedures that have been performed on the patient, medications taken by the patient, information about medical conditions of family members, and/or the like), and/or other information about the patient and/or the patient’s medical condition. AI agent 122 may be a software component that performs operations related to automated generation of content, such as retrieving/receiving relevant data, providing the relevant data along with a prompt to a machine learning model, and receiving an output from the machine learning model in response.

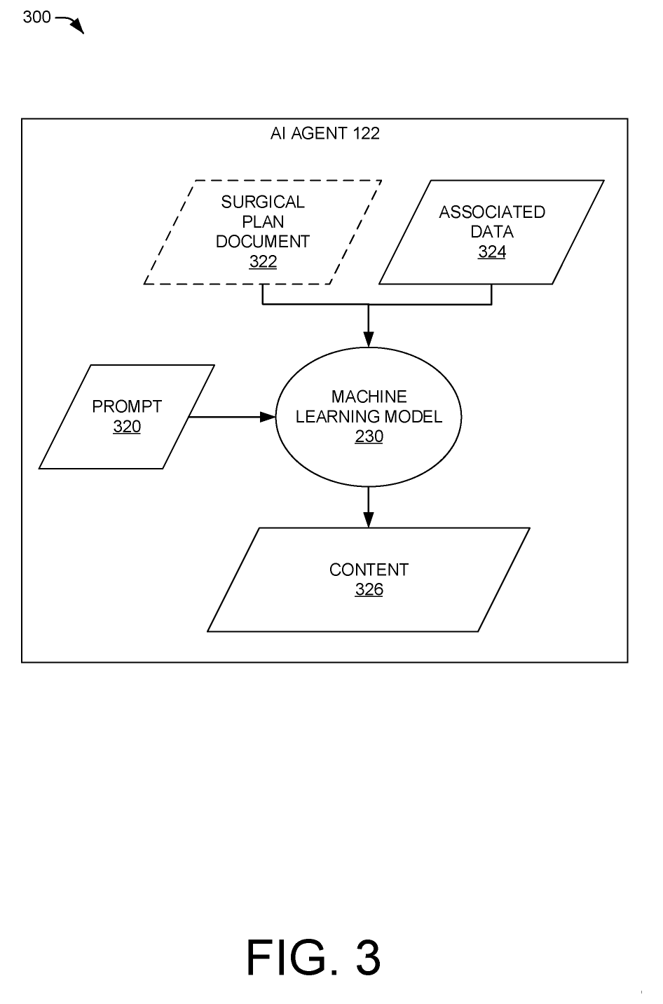

For instance, AI agent 122 may provide the retrieved data that is related to the request to the machine learning model along with a prompt that is based on the request, as described in more detail below with respect to FIG. 3. The prompt may, for example, be a natural language prompt instructing the machine learning model to output a listing of relevant patient information for a particular surgical procedure or to output indications of any automatically identified issues related to a particular surgical plan (e.g., that is defined in a surgical plan document provided to the model with the prompt) according to the request. In some cases the request itself is used as a prompt, while in other cases a prompt may be generated based on the request, such as automatically populating a prompt template based on the request, using a language processing machine learning model to automatically generate the prompt based on the request, using rules to automatically generate the prompt based on the request, and/or the like. The machine learning model may comprise a language processing machine learning model, one or more diffusion models capable of analyzing and/or generating audio, video, and/or image content, and/or the like. In some cases, the machine learning model may be a multimodal machine learning model.

Multimodal machine learning models such as multimodal large language models (MLLMs) transcend traditional text-based interfaces, and provide the ability to comprehend and generate content across a wide array of formats, including text, images, audio, and video. A multimodal machine learning model can integrate and interpret diverse forms of data, offering an unprecedented level of contextual understanding and interaction. For example, an AI agent may utilize such a model to perceive its environment based on various types of data in multiple modalities, thereby maximizing its chances of achieving its goals. In a surgical context, an AI agent can serve as a central hub, processing a wide array of data to facilitate efficient and effective surgical planning.

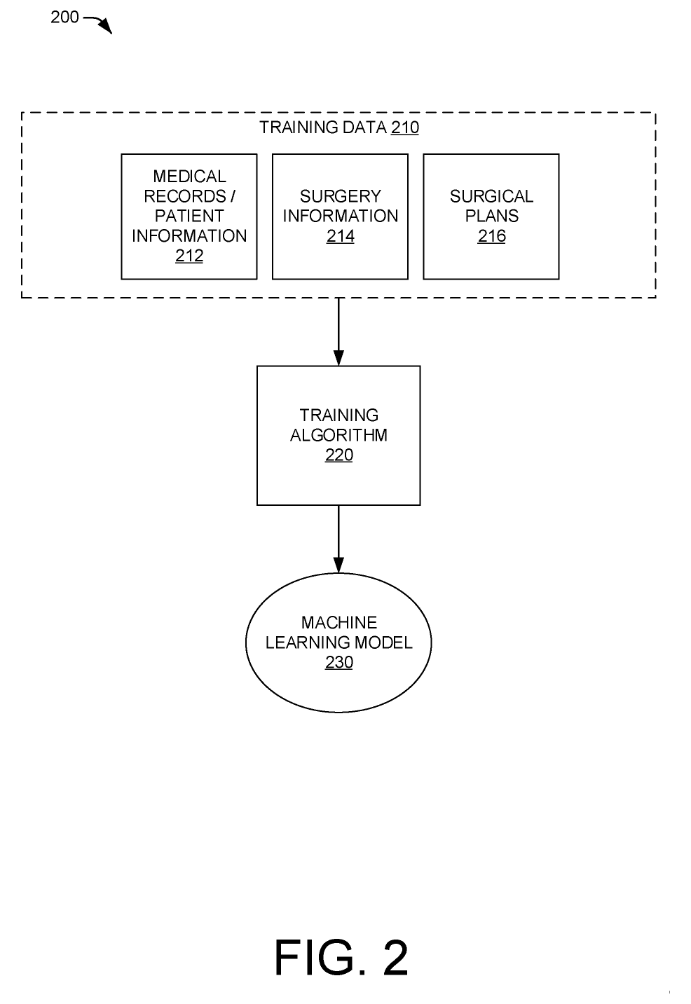

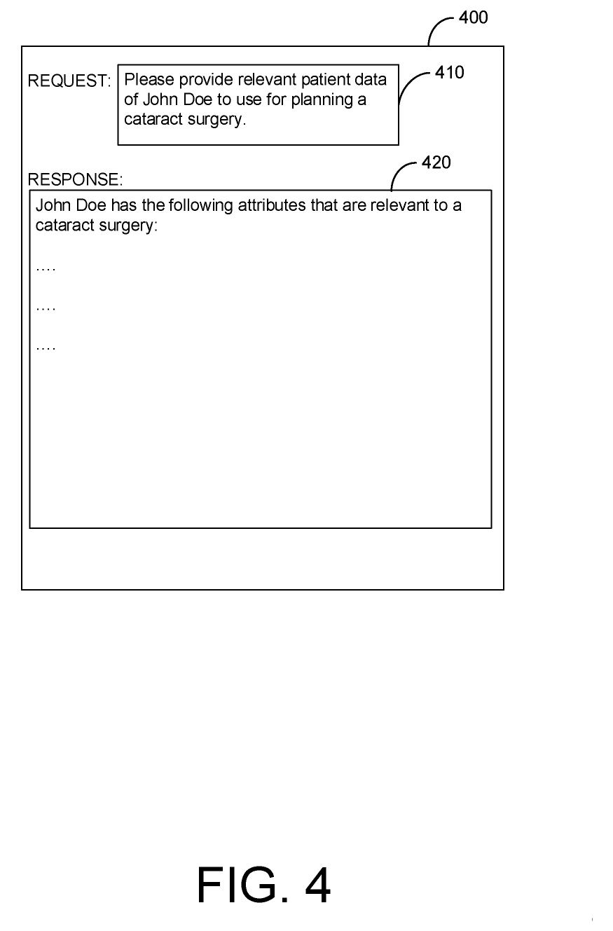

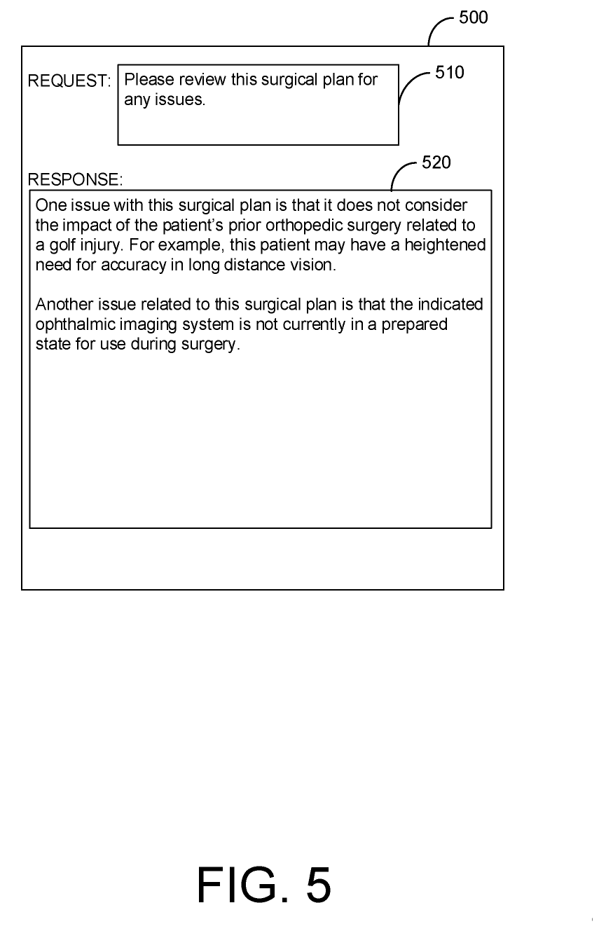

As described in more detail below with respect to FIG. 2, a machine learning model may have been fine tuned based on data specific to a domain in which it used, such as surgical planning. For example, the machine learning model may have been fine tuned based on historical medical data (e.g., from medical devices 160, 170, and/or 180) and/or other data to analyze and generate content based on such data. Certain examples of utilizing AI agent 122 to generate content in response to requests are explained in more detail below with respect to FIGS. 4 and 5.

A machine learning model may be trained or fine-tuned using supervised, semi-supervised, or unsupervised learning techniques. In some cases, more than one AI agent may be used. For example, different AI agents may use different machine learning models, such as of differing types and/or being trained using differing techniques, to generate content based on requests. For example, a first AI agent may use a machine learning model to generate lists of relevant patient information for a particular surgical procedure, while a different AI agent may use a different machine learning model to automatically analyze surgical plans and generate indication of issues related to such surgical plans. Multiple AI agents may run on AI server 120 and/or on one or more other devices. For example, techniques described herein with respect to AI agent 122 may also be used for one or more other AI agents.

Content generated by AI agent 122, such as content generated in response to a request, may be provided via digital output device 150. For example, text, image, video, and/or audio content may be output via digital output device 150 to one or more medical professionals.

In a typical operating room, there is a vast amount of data being generated and utilized. This may include information from medical equipment, human communication, pre-operative data, human movement, and/or the like. An AI agent according to techniques described herein can process all these types of data, providing valuable insights and assistance to the medical team, such as upon request, to assist in surgical planning. For example, medical equipment such as diagnostic instruments, electronic medical records (EMR) systems, monitors, ventilators, and surgical instruments generate and/or store a wealth of data. An AI agent can receive these data streams and utilize them to generate useful outputs to assist in surgical planning.

FIG. 2 illustrates an example 200 related to training a machine learning model for machine learning based surgical planning optimization, in accordance with certain embodiments of the present disclosure. Example 200 includes a machine learning model 230, which may be utilized by AI agent 122 of FIG. 1.

In example 200, training data 210 is used by a training algorithm 220 to train or fine tune machine learning model 230. Training data 210 includes (or is based on) medical records / patient information 212, surgery information 214, surgical plans 216, and/or the like. Medical records / patient information 212 generally include data about medical history and/or personal attributes of one or more patients, such as personal characteristics (e.g., age, gender, and/or the like), medical history (e.g., known medical conditions, information about the extent of known medical conditions, procedures that have been performed on the patient, medications taken by the patient, information about medical conditions of family members, and/or the like), data captured using diagnostic instruments, test results, treatment records, and/or other information about the patient and/or the patient’s medical condition. Surgery information 214 may include information about surgical procedures, such as being captured by one or more medical devices during performance of such procedures and/or including records of such activities, and/or data describing proper performance of and/or outcomes of such procedures, and/or the like. Surgical plans 216 may include, for example, surgical plan documents that were used to plan surgeries represented by surgery information 214 for patients represented by medical records / patient information 212. It is noted that the types of data depicted and described with respect to training data 210 are included as examples, and other types of data may also be included in training data 210.

Training algorithm 220 generally utilizes training data 210 to train machine learning model 230. For example, training algorithm 220 may involve supervised, unsupervised, or semi-supervised learning techniques by which machine learning model 230 is trained based on training data 210.

In some embodiments, labeled training data such as including sets of input features (e.g., prompts and subsets of medical records / patient information 212, surgery information 214, surgical plans 216, and/or the like) labeled with manually generated and/or manually validated content generated based on such input features is used in a supervised learning process to train machine learning model 230. In a typical supervised learning process, a set of training inputs is provided to a model, the model generates an output in response to the set of training inputs, the generated output is compared to a label associated with the training inputs, and one or more parameters of the model are adjusted based on the comparing, such as iteratively until one or more conditions are met. For instance, the one or more conditions may relate to an objective function (e.g., a cost function), or may relate to whether the outputs produced by the model based on the training inputs match the labels associated with the training inputs or whether a measure of error between training iterations is not decreasing or not decreasing more than a threshold amount. The conditions may also include whether a training iteration limit has been reached. Parameters adjusted during training may include, for example, hyperparameters, values related to numbers of iterations, weights, functions used by nodes to calculate scores, and the like. In some embodiments, validation and testing are also performed for a machine learning model, such as based on validation data and test data, as is known in the art.

In other embodiments, unsupervised learning techniques or semi-supervised leaning techniques are used during training algorithm 220 to train machine learning model 230 based on training data 210. For example, clustering techniques may be used to identify attributes in medical records / patient information 212 that are most highly correlated with positive or negative outcomes in surgery information 214 and/or to identify aspects of surgical plans 216 that are most highly correlated with positive or negative outcomes in surgery information 214. An outcome in surgery information 214 may be considered positive or negative based on input from one or more medical professionals and/or based on values falling within ranges or conditions associated with positive or negative outcomes, and/or the like. In some cases, semi-supervised learning may involve a certain amount of labeled training data that is compared to unlabeled training data to determine similarities (e.g., using clustering) so that labels applied to the labeled training data may also be associated with similar data in the unlabeled training data.

The training processes described above are included as examples, and other methods of training machine learning model 230 based on training data 210 are possible. In some embodiments, training algorithm 220 may involve one or more unsupervised learning processes (e.g., clustering), semi-supervised learning processes, and/or supervised learning processes. For example, unsupervised learning techniques or semi-supervised learning techniques may be used to analyze data and identify patterns. The results of such unsupervised and/or semi-supervised learning techniques may then be used in a supervised learning process, such as labeling input features for use in supervised learning based on such results. In other embodiments, labeled training data for a supervised learning process may be generated based on manual analysis of data and/or based on manual confirmation of results of an unsupervised learning process.

It is understood that a variety of machine learning techniques exist for such a training process, and any suitable machine learning algorithm(s) and/or model(s) may be used to train and/or fine tune machine learning model 230.

Machine learning model 130 may have been trained in advance of being fine tuned. For example, such pre-training may have been based on a large training data set that is more general in scope than training data 210, such as not being limited to a domain associated with training data 210. Techniques for training a machine learning model are known in the art.

Machine learning model 230 may, for example, be a language processing machine learning model such as a large language model (LLM), another type of generative machine learning model, a tree-based classification model, a neural network, a Bayesian network, a support vector machine, a multimodal large language model (MLLM), and/or the like. In one example, machine learning model 230 may include multiple models that are configured to analyze and/or generate different modalities. For example, multimodal machine learning model 230 may include an image input encoder, an audio input encoder, and a video input encoder that generate embeddings of image, audio, and video data, respectively. Such encoders may be used to convert different types of input data into embeddings that can be processed by a large language model (LLM) within a multimodal machine learning model, such as along with text data that can also be processed in embedding form by such an LLM. The LLM may be able to output embeddings of text, images, audio, and video, and machine learning model 230 may also include one or more diffusion models for generating outputs in image, audio, and video form based on such embeddings output by the LLM. For example, machine learning model 230 may include an image diffusion model, an audio diffusion model, and a video diffusion model, each of which may generate outputs in a particular modality. Machine learning model 230 may be capable as a result of its training and/or fine tuning of automatically generating outputs in one or more modalities in response to prompts based on inputs of varying modalities. Machine learning model 230 can utilize many specific expert models within its architecture to perform downstream tasks.

Furthermore, training algorithm 220 may be used to re-train machine learning model 230 as new training data becomes available.

FIG. 3 illustrates an example 300 of an artificial intelligence agent for machine learning based surgical planning optimization, in accordance with certain embodiments of the present disclosure.

In example 300, AI agent 122 of FIG. 1 provides a prompt 320 along with associated data 324 (which may include data retrieved from one or more devices such as medical devices 160, 170, and/or 180 of FIG. 1) to machine learning model 230 of FIG. 2, which outputs content 326 in response. Prompt 320 may be based on a request, such as input by a medical professional via an input device, for a particular type of information or other content. For example, the request may be for relevant information related to a patient for a particular surgical procedure or for automated analysis of a surgical plan. Prompt 320 may comprise the request, and may be provided to AI agent 122 along with associated data 324 as context. In some embodiments, such as if the request is for automated analysis of a surgical plan, a surgical plan document 322 may also be provided to machine learning model 230 along with prompt 320 (and, in some cases, along with other associated data 324). AI agent 122 may have retrieved associated data 324 (and/or, in some cases, surgical plan document 322) based on the request, such as based on comparing an embedding of the request to embeddings of various data items (e.g., from one or more medical devices) to determine which data items are relevant to the request (e.g., based on cosine similarity or another vector similarity comparison between the embedding of the request and the embeddings of data items). Associated data 324 may include data items determined based on such a comparison to be relevant to the request, such as having embeddings within a threshold Euclidean distance from the embedding of the request. Associated data 324 may include text, image data, video data, audio data, and/or the like. In some cases, surgical plan document 322 may be provided by or otherwise identified by the user that provided the request.

Machine learning model 230 may analyze associated data 324 and/or surgical plan document 322 based on prompt 320, and may generate content 326 based on such analysis. For example, content 326 may include text content, image content, video content, and/or audio content that was requested in prompt 320. In one example, content 326 includes a listing of patient data (e.g., from associated data 324) that is relevant to a particular surgical procedure indicated in the request. In another example, content 326 includes indications of one or more issues that were automatically identified by machine learning model 230 in surgical plan document 322. In some cases, content 326 may include one or more recommended changes to surgical plan document 322, such as to address one or more issues.

FIG. 4 illustrates an example related to machine learning based surgical planning optimization, in accordance with certain embodiments of the present disclosure.

In the depicted example, a screen 400 includes a request 410 and an associated response 420. For instance, screen 400 may represent a user interface screen and/or may otherwise represent a request received via an input device (e.g., via text input, audio input, video input, touch input mouse input, and/or the like) and a response (e.g., content) provided via an output device (e.g., a screen, a speaker, and/or the like).

Request 410 includes the language “Please provide relevant patient data of John Doe to use for planning a cataract surgery?” and may have been input by a medical professional prior to performing an ophthalmic surgical procedure. Request 410 may be processed by an AI agent as described above with respect to FIGS. 1 and 3, such as in connection with associated data, and the AI agent may automatically generate (e.g., using a machine learning model) response 420 based on the request and associated data. The associated data may include, for example, patient attributes, patient medical data, information about the surgical procedure that is being planned (e.g., cataract surgery), data about the patient that was captured via one or more medical devices and/or otherwise stored, live data about the patient’s current condition, inventory and/or stock information for items within the clinical facility, device state information of one or more devices, and/or the like.

Response 420 includes a natural language response to request 410, informing the medical processional that the patient John Doe has a particular set of attributes that are relevant to a cataract surgery, such as listing those relevant attributes. Response 420 may, as a result of being generated by a machine learning model that was trained based on training data indicating past correlations between particular patient attributes and particular surgical outcomes, accurately identify aspects of the patient’s data that are most relevant to the particular surgical procedure in a way that would not be possible with prior techniques.

FIG. 5 illustrates another example related to machine learning based surgical planning optimization, in accordance with certain embodiments of the present disclosure.

In the depicted example, a screen 500 includes a request 510 and an associated response 520. For instance, screen 500 may represent a user interface screen and/or may otherwise represent a request received via an input device (e.g., via text input, audio input, video input, touch input mouse input, and/or the like) and a response (e.g., content) provided via an output device (e.g., a screen, a speaker, and/or the like).

Request 510 includes the language “Please review this surgical plan for any issues” and may have been input by a medical professional along with an indication of a surgical plan (e.g., an identifier of a surgical plan document) prior to performing a surgical procedure. Request 510 may be processed by an AI agent as described above with respect to FIGS. 1 and 3, such as in connection with associated data, and the AI agent may automatically generate (e.g., using a machine learning model) response 520 based on the request and associated data. The associated data may include, for example, the surgical plan document, medical data associated with the patient, device state information, item inventory information, operating room availability information, and/or the like.

Response 520 includes a natural language response to request 510, informing the medical processional that “one issue with this surgical plan is that it does not consider the impact of the patient’s prior orthopedic surgery related to a golf injury. For example, this patient may have a heightened need for accuracy in long distance vision.” Response 520 further informs the medical professional that “another issue related to this surgical plan is that the indicated ophthalmic imaging system is not currently in a prepared state for use during surgery.” For example, the machine learning model may have determined, as a result of being trained based on historical surgical data, that the patient’s prior surgery related to a golf injury (e.g., indicated in the patient’s medical data) should be associated with one or more particular surgical plan features that are not present in the current surgical plan in order to achieve an optimal surgical outcome for the patient. Furthermore, the machine learning model may have determined, based on device state information, that an ophthalmic imaging system that is included in the surgical plan as a device that is to be used during the ophthalmic surgery is not currently in a prepared state.

These examples are included for explanation purposes, and many other examples are possible.

The issues indicated in response 520 may allow the surgical plan to be updated to overcome the issues and optimize the surgical outcome for the patient. In some aspects, response 520 may also include recommended changes to the surgical plan in order to overcome the identified issues. For instance, the machine learning model may be prompted by the AI agent to generate such recommendations, and the machine learning model may be able to generate such recommendations as a result of being trained on historical surgery data (e.g., indicating surgical plans that resulted in optimal surgical outcomes for patients having particular attributes and/or under particular contextual conditions).

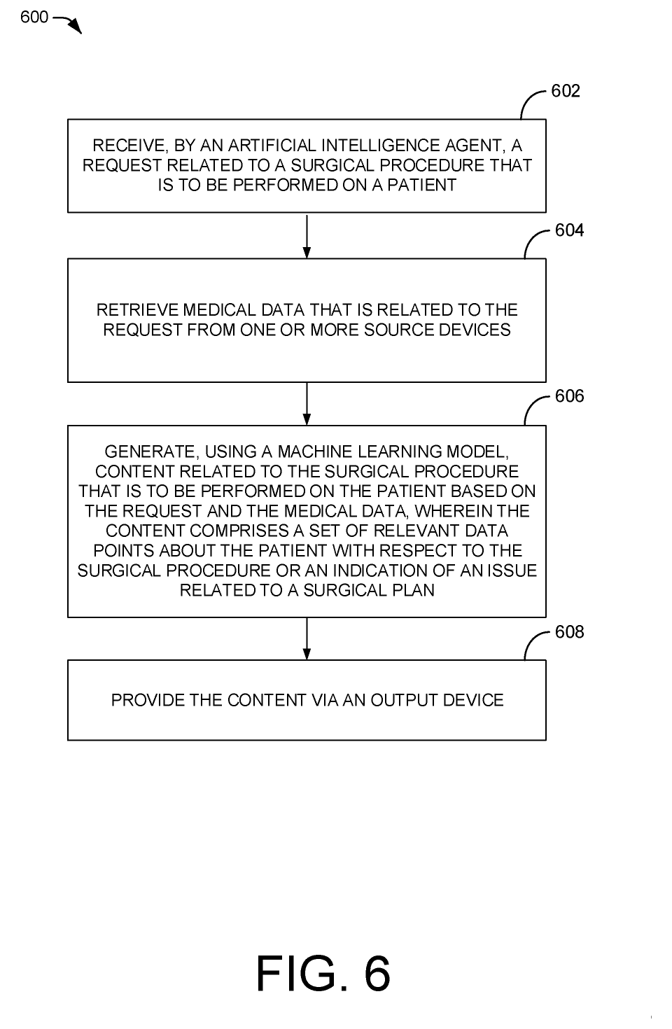

FIG. 6 illustrates an example of a process 600 related to machine learning based surgical planning optimization, in accordance with certain embodiments of the present disclosure. In certain embodiments, the process 600 can be implemented by one or more components described above with respect to FIGS. 1-3 and/or below with respect to FIG. 6. It is noted that any number of systems, in whole or in part, can implement the process 600.

Process 600 begins at block 602, with receiving, by an artificial intelligence agent, a request related to a surgical procedure that is to be performed on a patient.

Process 600 continues at block 604, with retrieving medical data that is related to the request from one or more source devices.

Process 600 continues at block 606, with generating, using a machine learning model, content related to the surgical procedure that is to be performed on the patient based on the request and the medical data, wherein the content comprises a set of relevant data points about the patient with respect to the surgical procedure or an indication of an issue related to a surgical plan.

Process 600 continues at block 608, with providing the content via an output device.

In some aspects, the content comprises the indication of the issue related to the surgical plan, and the issue related to the surgical plan relates to a machine state preparedness for the surgical procedure.

In certain aspects, the artificial intelligence agent determines the machine state preparedness for the surgical procedure by analyzing state data from a machine related to the surgical plan to determine whether the machine is prepared for performing its function for the surgical procedure.

In some aspects, the one or more source devices comprise one or more of: a diagnostic instrument; or an electronic medical record system.

In certain aspects, the issue related to the surgical plan comprises one or more of: a documentation error; a data entry inconsistency; an error in an intraocular lens power calculation; or an intraoperative data error.

In some aspects, the surgical procedure comprises one or more of: a cataract surgery; a minimally invasive glaucoma surgery; a vitreo-retinal surgery; or an intravitreal drug administration.

In certain aspects, the medical data comprises a surgical plan document.

In some aspects, the machine learning model has been fine tuned based on records related to past surgical procedures.

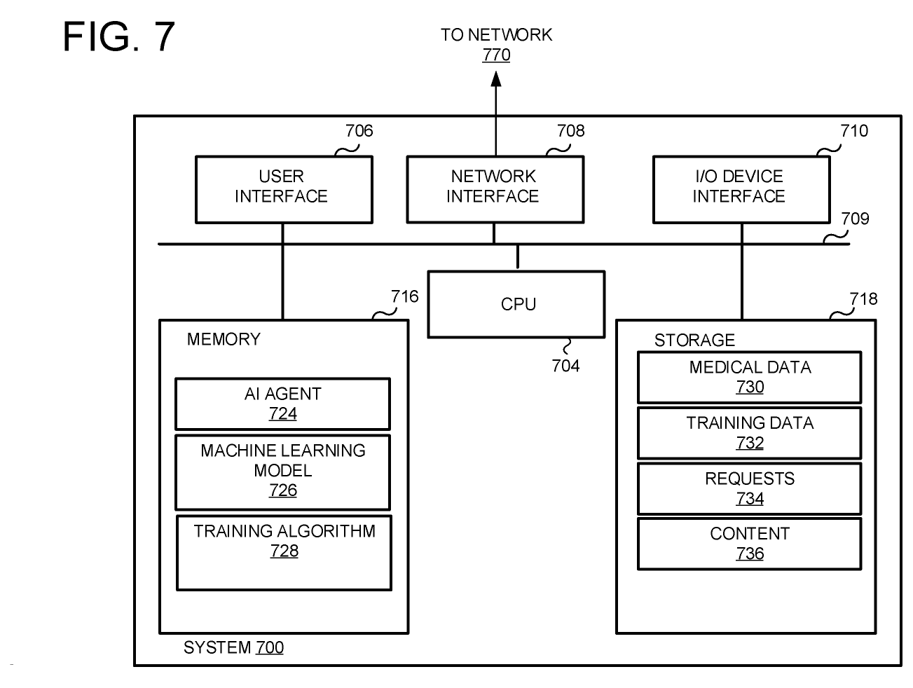

FIG. 7 illustrates an example of a system 700 for machine learning based surgical planning optimization, in accordance with certain embodiments of the present disclosure. For example, system 700 may be configured to perform method 600 of FIG. 6 and/or other aspects of the present disclosure, such as discussed above with respect to FIGS. 1-5.

As shown, system 700 includes, without limitation, central processing unit (CPU) 704, user interface 706, network interface 708, memory 716, storage 718, interconnect 709, and at least one I/O device interface 710 which may allow for the connection of various I/O devices (e.g., keyboards, displays, mouse devices, pen input, etc.) to system 700. While one or more operations are described herein as being performed by particular components of system 700, those operations may, in some embodiments, be performed by other components of system 700 and/or component(s) of other system(s). As an example, while one or more operations are described herein as being performed by CPU 704, memory 716, and/or storage 718 those operations may, in other embodiments, be performed by other components of system 700 or of a different system.

CPU 704 may be representative of one or more processing devices and/or cores. In some embodiments, CPU 704 may retrieve and execute programming instructions stored in memory 716. Similarly, CPU 704 may retrieve and store application data residing in memory 716. Interconnect 709 transmits programming instructions and application data, among CPU 704, I/O device interface 710, user interface 706, memory 716, storage 718, network interface 708, etc. In some embodiments, CPU 704 may correspond to a single CPU, multiple CPUs, or a single CPU having multiple processing cores. Additionally, in some embodiments, memory 716 represents volatile memory, such as random-access memory. In some embodiments, storage 718 may be non-volatile memory, such as a disk drive, solid state drive, or a collection of storage devices distributed across multiple storage systems.

System 700 can include a network interface 708 for connection with a data communications network (e.g., network 770), such as to communicate with other devices. The data communications network can be, or can include, one or more of a private network, a public network, a local or wide area network, the Internet, combinations of the same, and/or the like. The data communications network can include, for example, interfaces (e.g., application programming interfaces) for enabling interaction and communication between and among the components and systems of the computing environment (e.g., of FIG. 3) and/or other components and systems.

The memory 716 can include an AI agent 724, which generally represents AI agent 122 of FIG. 1. AI agent 724 may make use of a machine learning model 726 that is also depicted in memory 716, such as to automatically generate content 736 based on requests 734. For example, machine learning model 726 may be representative of machine learning model 230 of FIGS. 2 and 3. Memory 716 further comprises a training algorithm 728, which may be representative of training algorithm 220 of FIG. 2 In other embodiments, machine learning model 726 may be trained and/or fine tuned on a separate system from the system (e.g., system 700) on which the trained model is used to generate content.

The storage 718 can include medical data 730, which may be representative of medical data 164, 174, and/or 184 of FIG. 1. The storage 718 can also include training data 732, which may be representative of training data 210 of FIG. 2. The storage 718 can also include requests 734, which may be representative of prompt 320 of FIG. 3, request 410 of FIG. 4, and/or request 510 of FIG. 5. The storage 718 can also include content 736, which may be representative of content 326 of FIG. 3, response 420 of FIG. 4 and/or response 520 of FIG. 5.

It is noted that system 700 is included as an example, and techniques described herein may be implemented via fewer or more components, either on the same or different devices, and devices may include physical and/or virtual devices.

As used herein, a phrase referring to “at least one of” a list of items refers to any combination of those items, including single members. As an example, “at least one of: a, b, or c” or “at least one of: a, b, and c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A system for machine learning based surgical plan optimization, the system comprising:
one or more source devices configured to store or generate medical data related to a patient;
one or more interface devices configured to receive a request related to a surgical procedure that is to be performed on a patient; and
an artificial intelligence agent configured to:
retrieve a subset of the medical data that is related to the request from a subset of the one or more source devices; and
generate, using a machine learning model, content related to the surgical procedure that is to be performed on the patient based on the request and the subset of the medical data, wherein the content comprises a set of relevant data points about the patient with respect to the surgical procedure or an indication of an issue related to a surgical plan.

2. The system of claim 1, wherein the content comprises the indication of the issue related to the surgical plan, and wherein the issue related to the surgical plan relates to a machine state preparedness for the surgical procedure.

3. The system of claim 2, wherein the artificial intelligence agent determines the machine state preparedness for the surgical procedure by analyzing state data from a machine related to the surgical plan to determine whether the machine is prepared for performing its function for the surgical procedure.

4. The system of claim 1, wherein the one or more source devices configured to store or generate the medical data related to the patient comprise one or more of: a diagnostic instrument; or an electronic medical record system.

5. The system of claim 1, wherein the issue related to the surgical plan comprises one or more of:
a documentation error;
a data entry inconsistency;
an error in an intraocular lens power calculation; or
an intraoperative data error.

6. The system of claim 1, wherein the surgical procedure comprises one or more of:
a cataract surgery;
a minimally invasive glaucoma surgery;
a vitreo-retinal surgery; or
an intravitreal drug administration.

7. The system of claim 1, wherein the subset of the medical data comprises a surgical plan document.

8. The system of claim 1, wherein the machine learning model has been fine tuned based on records related to past surgical procedures.

9. A computer-implemented method of machine learning based surgical planning optimization, the computer-implemented method comprising:
receiving, by an artificial intelligence agent, a request related to a surgical procedure that is to be performed on a patient;
retrieving medical data that is related to the request from one or more source devices;
generating, using a machine learning model, content related to the surgical procedure that is to be performed on the patient based on the request and the medical data, wherein the content comprises a set of relevant data points about the patient with respect to the surgical procedure or an indication of an issue related to a surgical plan; and
providing the content via an output device.

10. The computer-implemented method of claim 9, wherein the content comprises the indication of the issue related to the surgical plan, and wherein the issue related to the surgical plan relates to a machine state preparedness for the surgical procedure.

11. The computer-implemented method of claim 10, wherein the artificial intelligence agent determines the machine state preparedness for the surgical procedure by analyzing state data from a machine related to the surgical plan to determine whether the machine is prepared for performing its function for the surgical procedure.

12. The computer-implemented method of claim 9, wherein the one or more source devices configured to store or generate the medical data related to the patient comprise one or more of: a diagnostic instrument; or an electronic medical record system.

13. The computer-implemented method of claim 9, wherein the issue related to the surgical plan comprises one or more of:
a documentation error;
a data entry inconsistency;
an error in an intraocular lens power calculation; or
an intraoperative data error.

14. The computer-implemented method of claim 9, wherein the surgical procedure comprises one or more of:
a cataract surgery;
a minimally invasive glaucoma surgery;
a vitreo-retinal surgery; or
an intravitreal drug administration.

15. The computer-implemented method of claim 9, wherein the medical data comprises a surgical plan document.

16. The computer-implemented method of claim 9, wherein the machine learning model has been fine tuned based on records related to past surgical procedures.

17. A non-transitory computer-readable medium comprising instructions that, when executed via one or more processors of a computing system, cause the computing system to:
receive by an artificial intelligence agent, a request related to a surgical procedure that is to be performed on a patient;
retrieve medical data that is related to the request from one or more source devices;
generate, using a machine learning model, content related to the surgical procedure that is to be performed on the patient based on the request and the medical data, wherein the content comprises a set of relevant data points about the patient with respect to the surgical procedure or an indication of an issue related to a surgical plan; and
provide the content via an output device.

18. The non-transitory computer-readable medium of claim 17, wherein the content comprises the indication of the issue related to the surgical plan, and wherein the issue related to the surgical plan relates to a machine state preparedness for the surgical procedure.

19. The non-transitory computer-readable medium of claim 18, wherein the artificial intelligence agent determines the machine state preparedness for the surgical procedure by analyzing state data from a machine related to the surgical plan to determine whether the machine is prepared for performing its function for the surgical procedure.

20. The non-transitory computer-readable medium of claim 17, wherein the one or more source devices configured to store or generate the medical data related to the patient comprise one or more of: a diagnostic instrument; or an electronic medical record system.
